(19) United States
(12) Patent Application Publication
(10) Pub. No.: US 2025/0391552 A1
(43) Pub. Date: Dec. 25, 2025

(54) UNIFIED COMMUNICATION AND EQUIPMENT TRACKING MOBILE APPLICATION FOR MEDICAL ENVIRONMENTS

(71) Applicant: Stryker Corporation, Portage, MI (US)

(72) Inventors: Serina JUSTINIANO-PARLAVECCHIO, North Chili, NY (US); Alan LEVINE, Lewiston, NY (US)

(73) Assignee: Stryker Corporation, Portage, MI (US)

(21) Appl. No.: 19/245,212

(22) Filed: Jun. 20, 2025

Related U.S. Application Data
(60) Provisional application No. 63/662,788, filed on Jun. 21, 2024.

(51) Int. Cl. G16H 40/40; G16H 40/20; H04M 1/72403

(52) U.S. Cl. CPC G16H 40/40; G16H 40/20; H04M 1/72403

(57) ABSTRACT

A mobile application providing a unified system for tracking visual equipment and enhancing communication in medical environments. The mobile application features tools for designing OR layouts, tracking equipment by serial numbers, and real-time troubleshooting. The mobile application integrates various forms of communication into a single platform and offers customizable administrative access and secure data management, streamlining operations, improving communication, and increasing efficiency in healthcare settings.

### CROSS-REFERENCE TO RELATED APPLICATIONS

This application claims the benefit of U.S. Provisional Application No. 63/662,788, filed Jun. 21, 2024, the entire contents of which are incorporated herein by reference.

### FIELD

The present invention relates to mobile applications designed for healthcare environments, specifically for tracking medical equipment and enhancing communication within Operating Rooms (ORs) and associated facilities.

### BACKGROUND

The invention is intended to solve challenges in healthcare facilities related to equipment tracking. Healthcare facility employees frequently receive inquiries from sales representatives about equipment details and frequently encounter misunderstandings that hinder operational efficiency. Current methods lack the integration and real-time collaboration necessary for efficient operation.

Therefore, there is a need in the healthcare industry for a mobile application that implements a unified communication system. Further, there is a need for a mobile application that integrates real-time collaboration necessary for efficient operation.

### SUMMARY

Disclosed is a mobile application for visual equipment tracking allowing users to create personalized visual representations of OR layouts, including detailed equipment tracking by serial numbers and real-time collaborative troubleshooting within virtual ORs. The mobile application integrates administrative oversight, sales support, and technical assistance into a single platform, enhancing communication through visualization.

In some embodiments, users can create personalized OR layouts using an interior design app-like interface allowing for accurate medical equipment placement and tracking by serial numbers within a virtual environment.

In some embodiments, the mobile application includes a feature for urgent troubleshooting alerts, enabling users to notify the entire team for immediate support. The mobile application determines who to alert and prioritizes notifications based on urgency.

In some embodiments, the mobile application integrates text, voice, video, and drawing features into a unified platform, facilitating real-time collaboration across departments.

In some embodiments, the mobile application allows administrative and clinical specialists to customize accounts, finalize OR layouts, and manage equipment inventory visually, with secure data management features.

In some embodiments, the mobile application consolidates various forms of communication into a single platform and archives them for accountability and review. It includes algorithms for organizing, searching, and retrieving archived communications.

These and other objects, features, and advantages of the present invention will become readily apparent upon a review of the following detailed description of the invention, in view of the drawings and appended claims.

### BRIEF DESCRIPTION OF THE FIGURES

Various embodiments are disclosed, by way of example only, with reference to the accompanying schematic drawings in which corresponding reference symbols indicate corresponding parts, in which:

FIG. 1 illustrates a login page where users enter their email address and password to access the application;

FIG. 2 illustrates an accounts page displaying hospital accounts;

FIG. 3 illustrates a facility layout with color-coded areas representing different hospital sections, allowing users to tap on areas to view detailed layouts;

FIG. 4 illustrates an OR core layout with clickable sections representing individual operating rooms;

FIG. 5 illustrates a detailed 3D view of an OR;

FIG. 6 illustrates a detailed 3D view of an OR;

FIG. 7 illustrates a detailed view of an OR with equipment and storage areas labeled for easy identification;

FIG. 8 illustrates a flowchart showing the workflow for managing equipment, including moving items to storage and automatically triggering tasks;

FIG. 9 illustrates a menu with various categories which can be selected by a user;

FIG. 10 illustrates a menu with various categories which can be selected by a user; and,

FIG. 11 illustrates a menu with various categories which can be selected by a user.

### DETAILED DESCRIPTION

The following description is not intended to be limited to the particular methodology, materials, and modifications described and, as such, may, of course, vary. It is also understood that the terminology used herein is for the purpose of describing particular aspects only and is not intended to limit the scope of the claims.

Furthermore, it should be appreciated that the drawings are representative to illustrate the inventive concepts herein and may not be to scale.

Unless defined otherwise, all technical and scientific terms used herein have the same meaning as commonly understood to one of ordinary skill in the art to which this disclosure pertains. It should be understood that any methods, devices, or materials similar or equivalent to those described herein can be used in the practice or testing of the example embodiments.

It should be understood that the use of “or” in the present application is with respect to a “non-exclusive” arrangement unless stated otherwise. For example, when saying that “item x is A or B,” it is understood that this can mean one of the following: (1) item x is only one or the other of A and B; (2) item x is both A and B. Alternately stated, the word “or” is not used to define an “exclusive or” arrangement. For example, an “exclusive or” arrangement for the statement “item x is A or B” would require that x can be only one of A and B. Furthermore, as used herein, when referring to a set or group of items, for illustration (A, B, C) the term “at least one or more . . . and . . . ” such as in “at least one or more of A, B, and C” is intended to include any to all of the denoted set or group of items, i.e. it could include just one item from the set or group, it could include all of the items from the set or group, and it could include any other combination of the set or group of items that is greater than one item and less than all of the items, the illustrated example having three items meaning there are up to seven non-ordered combinations A, B, C, AB, AC, BC, ABC. Other numbers of items would have maximum combination possibilities calculated accordingly.

Moreover, as used herein, the phrases “comprises at least one of” and “comprising at least one of” in combination with a system or element is intended to mean that the system or element includes one or more of the elements listed after the phrase. For example, a device comprising at least one of: a first element; a second element; and, a third element, is intended to be construed as any one of the following structural arrangements: a device comprising a first element; a device comprising a second element; a device comprising a third element; a device comprising a first element and a second element; a device comprising a first element and a third element; a device comprising a first element, a second element and a third element; or, a device comprising a second element and a third element. A similar interpretation is intended when the phrase “used in at least one of:” is used herein.

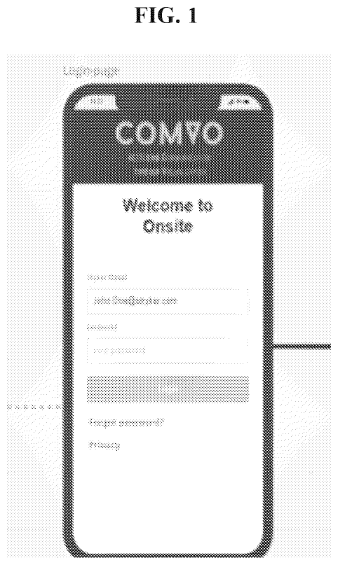

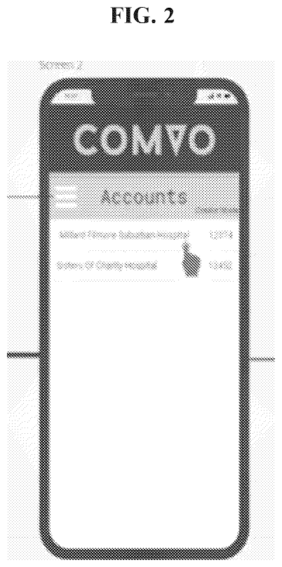

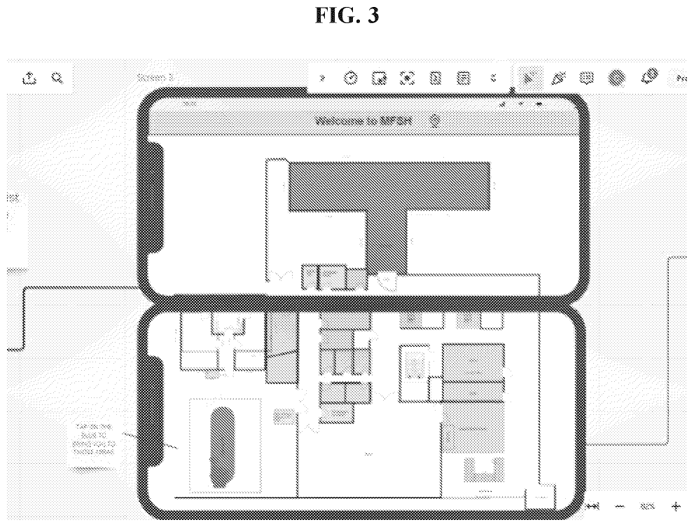

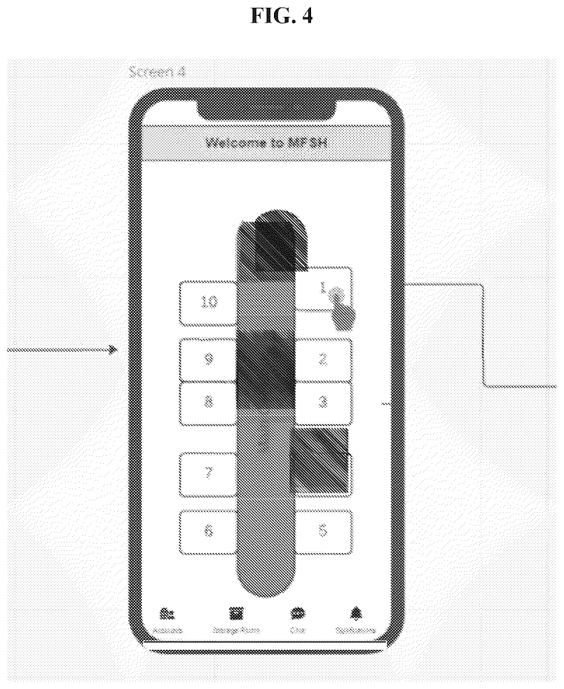

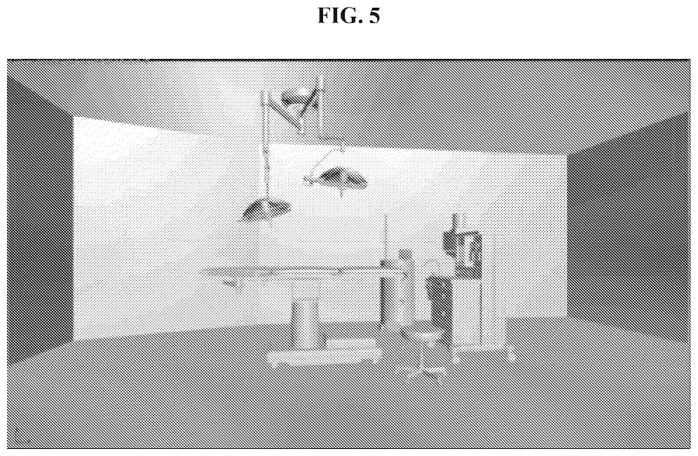

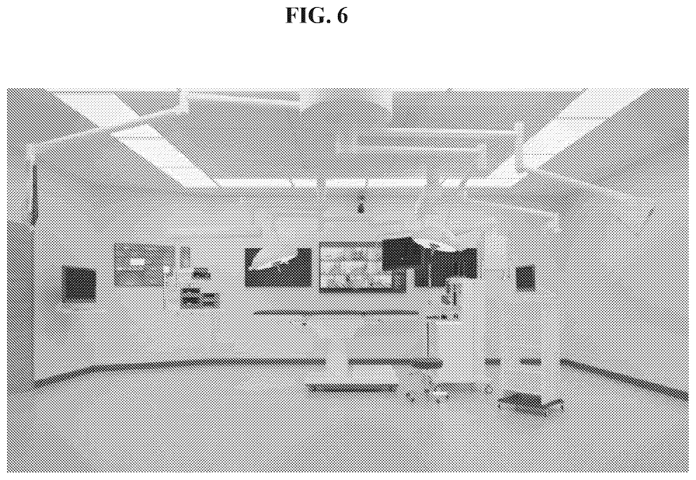

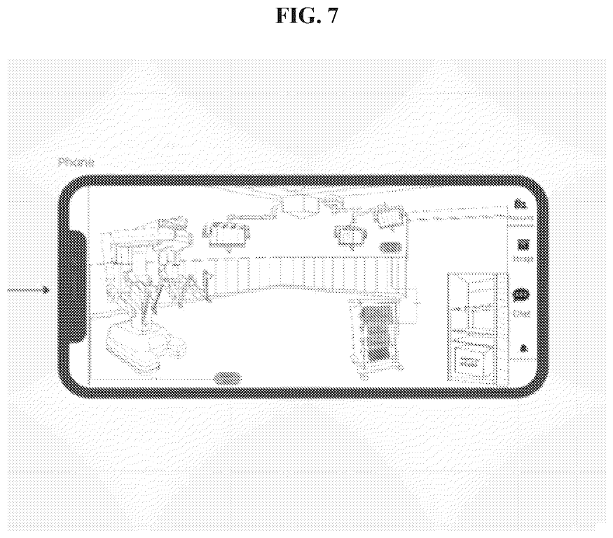

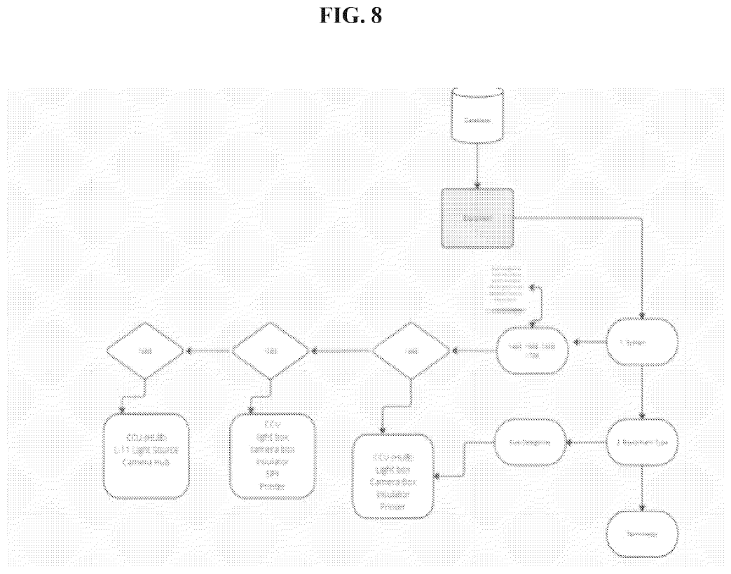

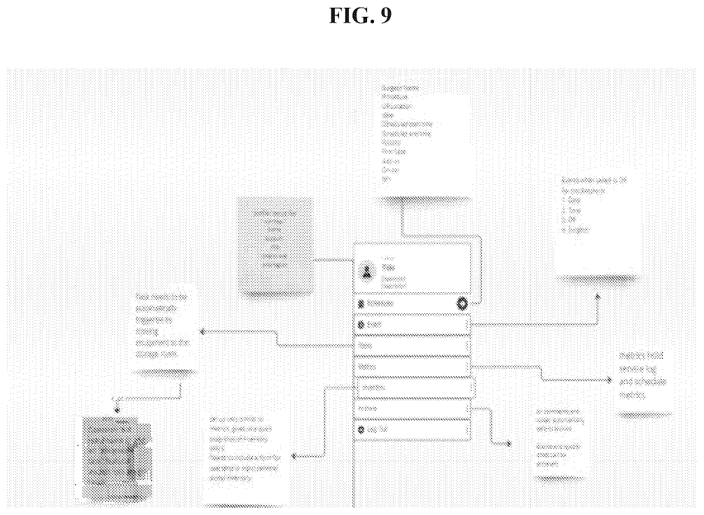

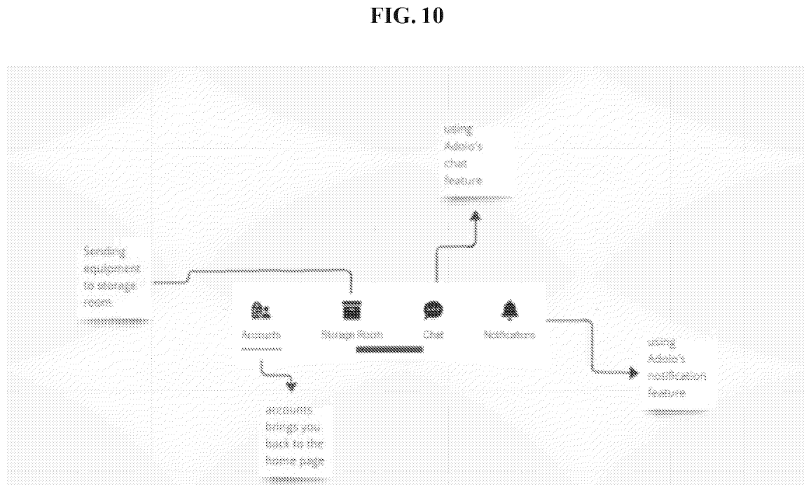

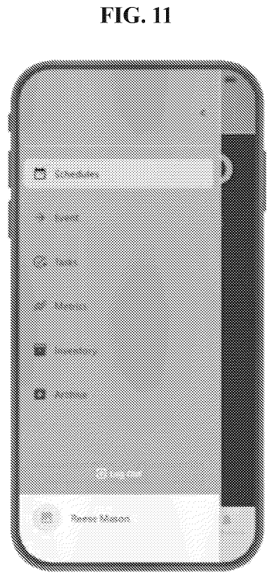

Disclosed in FIGS. 1-11 is a mobile application designed to streamline communication and equipment management in medical environments. The mobile application provides users with the ability to visually design OR layouts, place equipment accurately, and input serial numbers for comprehensive tracking. The mobile application supports real-time notifications for urgent troubleshooting, consolidates various communication forms, and offers customizable administrative access. The mobile application is developed using standard software development tools and key components include: a user interface for designing OR layouts and tracking equipment; backend algorithms for real-time notification and communication integration; a database for storing equipment details, user profiles, and communication logs; and security features for data management and account customization.

The mobile application is used by users logging into the mobile application and seeing their assigned accounts on the home screen. The user can navigate to specific OR layouts, manage equipment, and collaborate with team members in real-time. A troubleshooting button notifies the entire team for immediate assistance with urgent issues. Further, all interactions and changes are archived for future review.

### Customer Specifications

| Project Name | ComVo |
|---|---|
| Adalo Previewer | https://app.adalo.com/apps/e91fd9f0-7a38-4be4-97aa- |
| Link | ae4fe970aabf/screens |

### Product Specifications

| Admin | |
|---|---|
| Story | Log on, “Home Page- assigned accounts” |
| User | Morning Routine: |
| Story | Log In: Each day starts with specialists logging into the |
| | mobile application where they see their assigned |
| | accounts on the home screen. |
| | Account Access: The home screen also has a dropdown |
| | menu for quick access to specific features. At the top, |
| | there's a clickable profile with the specialist's picture, |
| | title, time in role, email, and phone number, similar to a |
| | workday profile. |
| | Essential Tabs: At the bottom of the screen, there are tabs |
| | for Notifications, Chat, Storage Room, and Archive, |
| | ensuring easy navigation. |
| | During the Day: |
| | Navigating the App: Specialists click on their account to |
| | view an aerial map of the facility. The map is color- |
| | coded, highlighting important areas, operating areas, and |
| | storage rooms. Clicking on these areas takes the |
| | specialist to detailed views. |
| | OR Core Inventory: Once in the OR core, there's an |
| | inventory tally of all working core equipment. Specialists |
| | can click into each OR to see all the equipment, matching |
| | the real OR setup. |
| | Managing Equipment: From within an OR, specialists |
| | can log events directly. To adjust equipment placement, |
| | they can right-click and select “swap” to move |
| | equipment between ORs or to the storage room. |
| | Real-Time Notifications: Every time equipment is moved |
| | or adjusted, a notification is sent to team members, |
| | keeping everyone informed. |
| | Storage Room Management: |
| | Task Assignment: When an item is placed in the storage |
| | room, a task is created for the specialist. This task can be |
| | reassigned to other team members or all members, |
| | ensuring that the equipment is managed promptly. |
| | Sticky Notes: Specialists can leave sticky notes on all |
| | pages for reminders or instructions, which can later be |
| | moved to the archive. |
| | Communication and Troubleshooting: |
| | Real-Time Collaboration: If an urgent issue arises, the |
| | specialist can press the “troubleshoot” button, instantly |
| | notifying the entire team. They can collaborate via chat |
| | and click into the OR that a team member is in so the |
| | problem can be described. |
| | Unified Communication: The mobile application |
| | consolidates all communication forms into one platform, |
| | streamlining interactions across departments and |
| | improving response times. |
| | End of the Day: |
| | Review and Archive: All communications and changes |
| | are automatically archived. Specialists can review these |
| | logs to ensure accountability and prepare for the next |
| | day |
| Business | The mobile application introduces features for visual |
| Case | equipment tracking, akin to an interior design application. |
| | It allows users to create personalized, visual representations |
| | of Operating Room (OR) layouts. This visualization extends |
| | to detailed equipment tracking by serial numbers and real- |
| | time, collaborative troubleshooting within virtual ORs. The |
| | mobile application integrates administrative oversight, sales |
| | support, and technical assistance within a single platform, |
| | fostering better communication through visualization. |
| | Key Features: |
| | Visual Equipment Tracking: Users can visually |
| | design their OR layout, place equipment |
| | accurately, and input serial numbers for |
| | comprehensive tracking. |
| | Virtual OR Collaboration: An urgent |
| | troubleshooting button enables instant team |
| | notifications for assistance, enhancing the support |
| | system for onsite specialists. |
| | Enhanced Communication: The mobile |
| | application consolidates various forms of |
| | communication—text, voice, video, and notes—into |
| | a unified platform, streamlining interactions |
| | across departments. |
| | Customizable Administrative Access: The mobile |
| | application for administrative and clinical |
| | specialists allows for tailored account |
| | customization and secure data management. |
| | Visual Equipment Tracking and Virtual OR |
| | Layout Design |
| | The method and system for creating personalized |
| | Operating Room (OR) layouts using an interior |
| | design app-like interface allows users to |
| | accurately place and track medical equipment by |
| | serial numbers within a virtual environment. This |
| | could include the specific algorithms or software |
| | mechanisms that enable such detailed |
| | customization and visualization. |
| | Urgent Troubleshoot Notification System |
| | The design and implementation of an urgent |
| | troubleshooting notification system within the |
| | mobile application allows users to alert the entire |
| | team for immediate support instantly. The novelty |
| | might lie in how the mobile application |
| | determines who to alert, prioritizes alerts, and |
| | how users interact with the notification system. |
| | Integrated Communication Platform |
| | A comprehensive communication system that |
| | integrates text, voice, video, and drawing features |
| | into a unified platform for real-time collaboration |
| | among various departments could be patentable. |
| | Particularly patentable aspects include seamlessly |
| | integrating these different forms of |
| | communication and the user interface design that |
| | facilitates such integration. |
| | Administrative and Customization Features |
| | The system allows specialists to customize their |
| | accounts, lock and finalize OR layouts, and |
| | manage equipment inventory visually, including |
| | specific user interface elements or software |
| | processes that enable such customization and |
| | security features. |
| | Hybrid Communication and Documentation Archive System |
| | The process of consolidating communication |
| | from various channels (text, voice, video, notes) |
| | into a single mobile application and archiving |
| | them for accountability and review purposes. This |
| | could extend to the algorithms for organizing, |
| | searching, and retrieving archived |
| | communications based on various parameters. |

### Roles and Features

| Role | Feature | Desired functionality |
|---|---|---|
| Administration | Registration | Sign up and register in mobile |
| | | application as proper role, assign |
| | | account. |
| Specialist/ all | Set Profile | Should be able to set profile information |
| | | (name, picture) role, time in role, chat |
| | | information. |
| All | Map | Created on smart draw—add hyper links |
| | | to the blue areas that should be |
| | | clickable—changing the view to those |
| | | specific locations. |
| All | Notifications | Users should receive real-time |
| | | notifications for equipment movement, |
| | | task assignments, and urgent |
| | | troubleshooting alerts. |
| Specialist | Create ORs | Specialists should be able to create OR |
| | | layouts, place equipment, and input |
| | | serial numbers for tracking. |
| Specialist | Storage room | When an item is placed in the storage |
| | | room, a task should be created and |
| | | assigned to ensure it is managed |
| | | promptly. |
| All | archive | All communications and changes should |
| | | be automatically archived for review |
| | | and accountability. |

| Role | Feature | Desired functionality |
|---|---|---|
| All | chat | Integrated chat functionality for real- |
| | | time communication between users. |
| | Troubleshoot | A dedicated button for urgent |
| | button | troubleshooting alerts, notifying the |
| | | entire team immediately. |
| Sales | Account | Sales representatives can view all |
| Division | Overview | their accounts on their phone, including |
| | | detailed equipment tracking and status. |
| Sales | Communication | Ability to communicate with specialists |
| Division | | and review notes related to accounts and |
| | | equipment. |
| Sales | Issue Tracking | View and understand any issues with |
| Division | | equipment, and track resolutions in real |
| | | time. |
| Tech | Real-Time Chat | Specialists can chat with tech support in |
| Support | | real-time to resolve issues quickly. |
| Tech | Screen Drawing | Specialists can draw on the screen to |
| Support | | illustrate problems directly to tech |
| | | support. |
| Customer | Equipment | Customers can view the status and |
| | Status | location of their equipment. |
| Customer | Specialist | Ability to communicate directly with |
| | Communication | specialists to ask questions or report |
| | | issues. |
| Customer | Documentation | Access to relevant documentation, |
| | Access | manuals, and notes related to their |
| | | equipment. |

### Database Collections

### Users

### Fields:

Email address (text)
Password (text)
Username (text)
Full Name (text)
Phone Number (number)
Profile Image (image)
Role (text)—to distinguish between admin, specialist, sales, tech support, customer.
Time in Role (text)
Chat Information (text)

### Accounts

### Fields:

Account Name (text)
Account Number (number)
Assigned Users (relationship to Users)—to link users to specific accounts.

### Equipment

### Fields:

Name (text)
Model (text)
Sticker Label Number (text)
Serial Number (number)
Located (text)—to specify the current location.
Last Moved (date)
OR (relationship to OR Layouts)—to link equipment to specific ORs

### OR Layouts

### Fields:

Name (text)
Map Image (image)
Equipment (relationship to Equipment)—to list equipment in the OR
Facility (relationship to Accounts)—to link OR layouts to specific accounts-Tasks

### Fields:

Task Description (text)
Assigned To (relationship to Users)
Status (text)—e.g., pending, completed
Created At (date)
Due Date (date)
Notifications

### Fields:

Message (text)
Type (text)—e.g., equipment movement, task assignment, troubleshooting alert
Related User (relationship to Users)
Created At (date)
Related Equipment (relationship to Equipment)—optional, for equipment-related notifications
Communications

### Fields:

Message Type (text)—e.g., text, voice, video, note
Content (text)
From User (relationship to Users)
To User (relationship to Users)
Timestamp (date)
Related Equipment (relationship to Equipment)—optional, for equipment-related communications
Archives

### Fields:

Communication (relationship to Communications)
Archived At (date)

### Example Data Flow

### User Logs In:

User logs in and sees their assigned accounts on the home screen. The system pulls user-specific data from the Users collection.

### Navigating Accounts:

User clicks on an account to view OR layouts (linked via the Accounts collection).
OR layouts display mapped equipment, pulled from the Equipment collection linked to the specific OR layout.

### Managing Equipment:

User moves equipment, which updates the Equipment collection with the new location and movement date.
Notifications are sent out, creating entries in the Notifications collection.

### Communication:

Users send messages, creating entries in the Communications collection. Messages can be archived, creating entries in the Archives collection linked to the original communications.

What is claimed is:

1. A method of tracking equipment in a healthcare facility using a mobile application, comprising:
creating a personalized visual representation of an Operating Room (OR) layout;
tracking equipment within said visual representation of said OR layout using serial numbers corresponding to said equipment;
virtually placing said equipment within said visual representation of said OR layout; and
receiving real-time notifications for troubleshooting said equipment.
